(19) United States
(12) Patent Application Publication
(10) Pub. No.: US 2020/0289156 A1
(43) Pub. Date: Sep. 17, 2020

(54) INTRODUCER AND METHODS OF USE THEREOF

(71) Applicant: The Feinstein Institute for Medical Research, Manhasset, NY (US)

(72) Inventors: Ernesto MOLMENTI, Great Neck, NY (US); Conor HAND, Dublin (IE); Derek YOUNG, Dublin (IE)

(21) Appl. No.: 16/086,050

(22) PCT Filed: Mar. 17, 2017
(86) PCT No.: PCT/US2017/023029

§ 371 Date: Sep. 18, 2018

Related U.S. Application Data
(60) Provisional application No. 62/334,344, filed on May 10, 2016.
(60) Provisional application No. 62/310,431, filed on Mar. 18, 2016.

(51) Int. Cl. A61B 17/34

(52) U.S. Cl. CPC A61B 17/3474; A61B 2017/00407; A61B 17/3498; A61B 17/3494

(57) ABSTRACT

Medical instrument introducers including instrument drive assemblies to incrementally advance a medical instrument are described. Methods of inserting an instrument into a patient, including incrementally advancing the instrument into the instrument introduction site, are also described.

### TECHNICAL FIELD

The present disclosure generally relates to devices and methods for the introduction of medical instruments or objects into a patient. Specifically, the present disclosure relates to devices and methods for controlled introduction of a medical instrument into a surgical site on a patient.

### BACKGROUND

Laparoscopic instruments are traditionally introduced via set ports that pass through the skin and fascia in order to access the desired operative site. The placement of such ports is traditionally achieved by first creating a pneumoperitoneum, and then inserting a port with a trocar or similar device.

Insertion of initial trocars and ports, subsequent trocars, needles, and other surgical tools can result in complications, such as port scars, hernias at insertion points, and trauma to the abdominal wall. The use of set ports during a surgery also limits the ability of a surgeon to select the optimal insertion point for an instrument, and requires larger incisions which the surgeon must suture or otherwise close after completion of the surgery. Additionally, existing methods of inserting medical instruments into a surgical field may result in accidental over insertion into the surgical site resulting in damage to internal structures.

The present disclosure overcomes one or more of these problems, and/or other problems in the art.

### SUMMARY

A method of inserting an instrument into a patient includes supplying pressurized fluid to an introducer to an instrument introduction site, and advancing an instrument located in the introducer through the introduction site after an initiation of supplying of pressurized fluid.

According to another aspect, a medical instrument introducer includes a housing having a proximal end and a distal end, and an instrument support supporting an instrument from the proximal end to the distal end. The introducer also includes an instrument drive assembly having a drive actuator to incrementally advance the instrument through the housing.

A medical instrument introducer includes a housing having a proximal end and a distal end, and an instrument support supporting an instrument from the proximal end to the distal end. The introducer also includes a pressurized fluid supply assembly, an instrument drive assembly having a drive actuator to incrementally advance the instrument through the housing, and an exit port at the distal end of the housing, the exit port receiving both the instrument and pressurized fluid from the pressurized fluid supply assembly.

It may be understood that both the foregoing general description and the following detailed description are exemplary and explanatory only and are not restrictive of the features claimed.

As used herein, the terms “comprises” “comprising,” or any other variation thereof, are intended to cover a non-exclusive inclusion, such that a process, method, article, or apparatus that “comprises” a list of elements does not necessarily include only those elements, but may include other elements not expressly listed or inherent to such process, method, article, or apparatus. The term “exemplary” is used in the sense of “example,” rather than “ideal.”

### BRIEF DESCRIPTION OF THE DRAWINGS

The accompanying drawings, which are incorporated in and constitute a part of this specification, illustrate various exemplary embodiments and together with the description, serve to explain the principles and operation of the disclosed embodiments. Any features of an embodiment described herein (e.g., device, method of treatment) may be combined with any other embodiment, and are encompassed by the present disclosure.

FIG. 1 shows a schematic view of an exemplary introducer device in accordance with the present disclosure.

FIG. 2 shows an exemplary drive assembly of the introducer device of FIG. 1.

FIG. 3A shows another exemplary drive assembly of the introducer device of FIG. 1.

FIG. 3B shows an exemplary introducer device.

FIG. 3C shows another exemplary introducer device.

FIG. 4 shows yet another exemplary drive assembly of the introducer device of FIG. 1.

FIG. 5 shows a partial cross section view of the distal end of the introducer device of FIG. 1.

FIG. 6 is a flow chart of exemplary steps of a method in accordance with the present disclosure.

FIGS. 7A-7C illustrate certain exemplary steps of the method of FIG. 6.

FIG. 8 shows yet another exemplary introducer device.

FIG. 9 shows yet another exemplary introducer device.

FIG. 10 shows yet another exemplary introducer device.

### DETAILED DESCRIPTION

The present application relates to embodiments of an introducer for the incremental introduction or retraction of an instrument into or from a desired location. The introducer could be used to introduce any appropriate instrument, for any type of application, in any environment. For example, the introducer could be used for the incremental introduction or retraction of a medical instrument into or from a surgical site on a patient (adult, pediatric, adolescent and/or geriatric). The embodiments provided herein will be explained with respect to the introduction of medical instruments, but it is understood that the introducer is not limited to such medical uses. The device as described may also be used in many existing veterinary procedures. Reference now will be made in detail to aspects of the present disclosure, examples of which are illustrated in the accompanying drawings. Wherever possible, the same reference numbers will be used throughout the drawings to refer to the same or similar parts.

The term “distal” refers to a portion farthest away from the operator when introducing an instrument into a subject. By contrast, the term “proximal” refers to a portion closest to the operator when placing the instrument into the subject. The following description refers to the introduction of medical instruments. As used herein, a “medical instrument” may include any type of instrument or device that is used in a medical procedure, regardless of the particular use purpose, specialty, or size of the instrument. For example, the medical instrument may be a laparoscopic instrument, such as a mini-laparoscopic instrument, a needle, sensor/chip, catheter, transfusion device, drain tube, biopsy needle, and/or devices containing fluids, etc. The medical instrument may also be an introduction port, such as a trocar, to provide an accessway for one or more additional medical instruments, or for the simultaneous introduction of instruments with the introduction port. Such an introduction port could be a regular port (such as 5, 10, 12 mm) or smaller. While reference is made in this specification to medical instruments having generally straight shafts, it is understood that bent or curved instruments may also be used with the disclosed introducers.

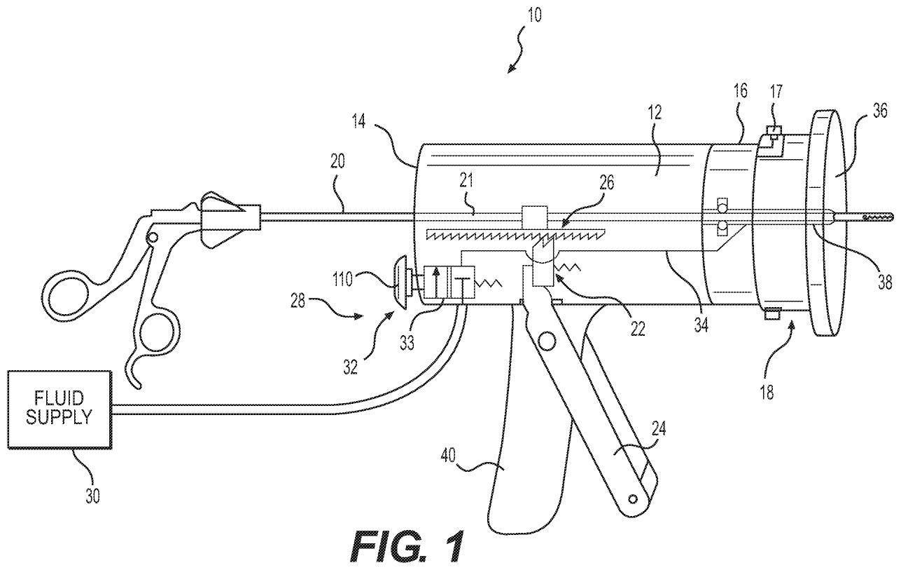

FIG. 1 shows a medical device introducer 10 in schematic form. The introducer 10 includes a housing 12 having a proximal end 14 and a distal end 16. A distal cap 18 may be located at the distal end 16 of the introducer 10. A medical instrument 20 is supported within the introducer 10 and is coupled to an instrument drive assembly 22. The instrument drive assembly 22 includes a drive actuator 24 and incremental drive linkages 26. The introducer may also include a fluid supply assembly 28, including a pressurized fluid supply 30, a valve assembly 32, and a fluid conduit 34 that may run to the distal end 16 of the housing 12. The distal cap 18 may include a coupling end 36 for coupling with the tissue of a patient, and a passage 38 for receiving the medical instrument 20 and a supply of fluid from the fluid supply assembly 28, for allowing both the medical instrument 20 and the pressurized supply of fluid to exit through a distal opening in the introducer 10. As shown in FIG. 1, the drive actuator 24 may extend on both sides of a handle 40. The valve assembly 32 may be provided at a central location of the proximal end 14 of the housing 12 so as to be positioned for use by both left and right handed users.

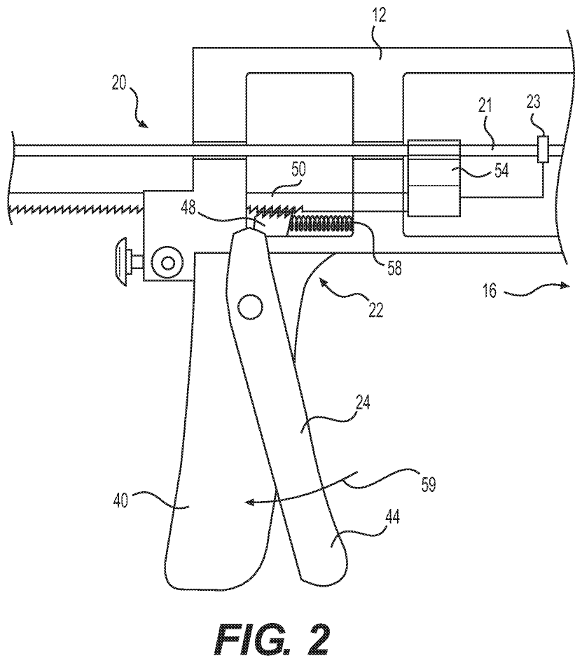

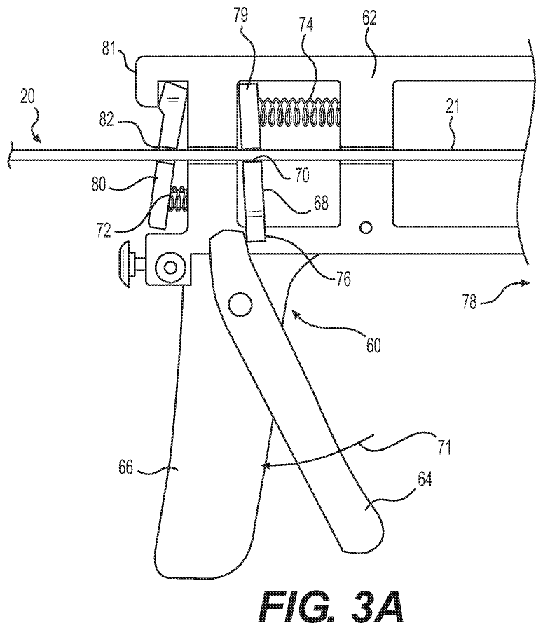

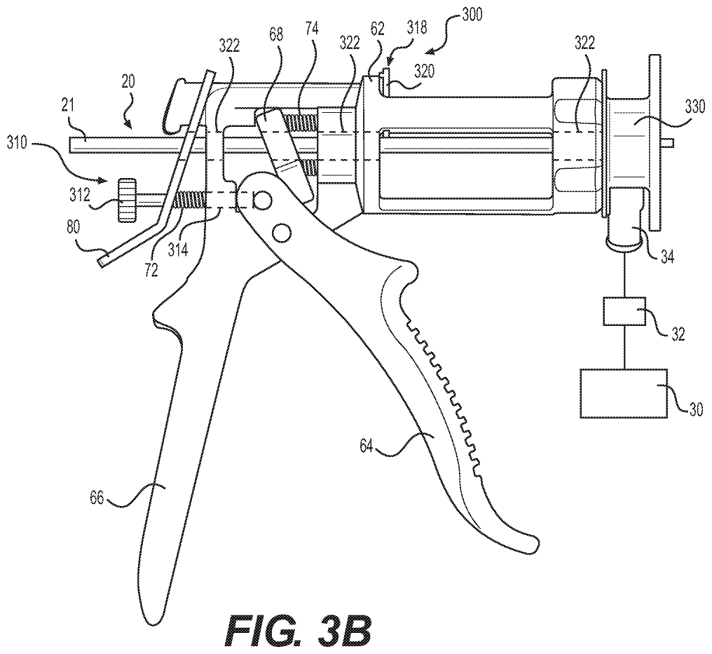

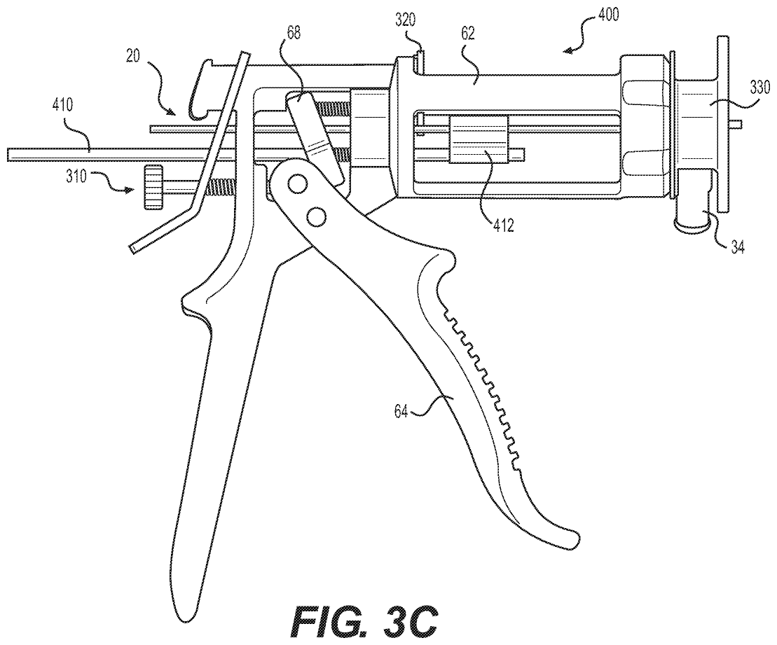

The housing 12 may receive a shaft of the medical instrument 20 as well as a part or all of the instrument drive assembly 22 and the fluid supply assembly 28. The housing 12 may be any of a variety of shapes and sizes. Housing 12 may include, for example, a cylindrical shape as shown in FIG. 1, or may have a rectangular open frame configuration (FIGS. 2 and 3) to allow for ease of loading and removal of the medical instrument 20 before, during, and/or after introduction. Thus, the housing 12 may fully enclose a portion of the medical instrument 20, or partially surround the medical instrument 20 with an open frame (FIGS. 2 and 3) to allow for quick removal of the medical instrument 20 from the housing 12. The shape of the housing 12 may have contouring, for example, to allow for ease of grip by a user, or to fit more ergonomically into a surgical field. The housing 12 may be made in a variety of sizes, based on, for example, the size of the medical instrument 20 to be inserted by the introducer 10, the size of the drive assembly 22, and/or ease of handling or use. In another embodiment using various inserts with the device, it may also be possible to use the same introducer device for various sizes of instrument. For example using an insert kit it may also be possible to use instruments from 2, 3, 4, 5 mm and including the larger size devices.

As best shown in FIGS. 3-5, the housing 12 may have one or more instrument supports to support the medical instrument 20 from the proximal end 14 to the distal end 16. The handle 40 of housing 12 may be configured as a stationary pistol grip, as depicted in FIG. 1. The housing 12 may optionally be constructed in multiple parts, with at least one openable seam running from the proximal end 14 to the distal end 16, such that it may be separated for accessing the medical instrument 20. Alternatively, the housing 12 could be a closed structure with a securable door member (not shown) that would allow for connection/access/removal of the medical instrument 20.

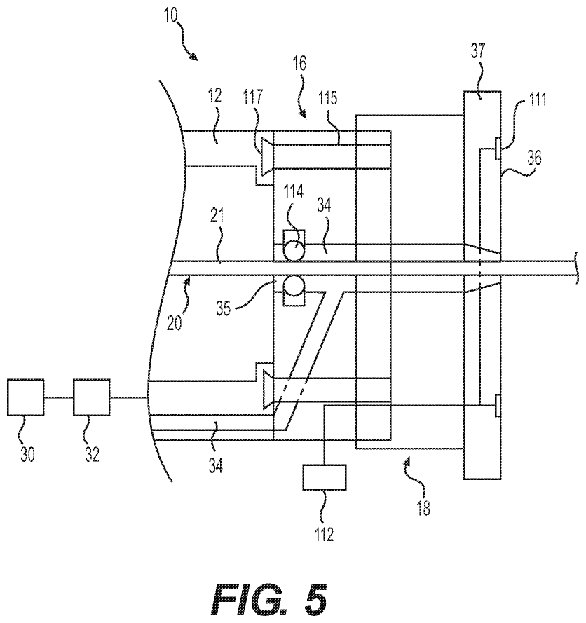

The proximal end 14 of the housing 12 may be located proximate to the user or operator of the introducer, and the distal end 16 may be located closest to the tissue of the subject. The distal cap 18 is located at the distal end 16 of introducer 10. The distal cap 18 may be constructed as a contiguous part of the distal end 16, or may be a separately constructed part. The distal cap 18 may be a single unit or may divide in order to allow for its removal after introduction of the medical instrument 20. The distal cap 18 may cover the entire distal end 16, and may be coupled to the distal end 16 either removably or permanently. For example, distal cap 18 may be removably connected to distal end 16 by a peg-in-groove rotational coupling 17 (FIG. 1), by a threaded connection, by adhesive, or otherwise. With such a removable distal cap 18, the housing 12 could be a reusable component while the distal cap 18 could be single-use only. Removal of the housing 12 from the medical instrument 20 may include any appropriate connection, for example, a series of dovetail connections 115 and 117 (FIG. 5). The proximal end 14 of a frame-type housing 12 (FIGS. 2 and 3) may be removed from the medical instrument 20 (and separated from housing distal end 16) by sliding the frame-type housing 12 along lateral dovetails connections 117 formed with distal end 16. Thereafter, the remaining distal end 16 of housing that encircles the medical instrument 20 can be removed from medical instrument 20 (after disconnection from distal cap 18 as described above) by sliding opposite mating portions of distal end 16 along axial dovetails 115. With such a removal process, seal 114 may remain on medical instrument 20 and be removed as desired. Alternatively, the distal cap 18 may be retained in position on the patient to provide support for the medical instrument 20, or for introduction of a different medical instrument 20.

The medical instrument 20 may be any medical instrument or device having an elongate shaft for introduction into a patient. Examples of suitable instruments 20 include laparoscopic instruments, laparoscopic cameras, trocars, cannulas, wires, interventional radiology devices, stents, stent introducers, substance introducers, catheters of all sizes and rigidities, or fluid tubes.

In another embodiment when introducing an instrument or device, the instrument or device could be surrounded by a sheath that would be advanced together with the instrument or device. This sheath could serve several functions, such as to allow for fluid to be delivered at the tip of the instrument or device being introduced, act as a port if left in place. This could potentially allow for the removal of the instrument and the subsequent re-introduction of the same or of another instrument or device. The sheath could also allow for the placement at its tip or on a tip of a medical instruments 20, one or more sensors to monitor, for example, pressure, or pH, etc. The sheath could also allow for the placement at its tip of a small blade or cauterization device that would incise/cut tissue with each advancement allowing for an easier introduction of the instrument or device. A sheath could also serve to protect the medical instrument 20, such as a laparoscopic grasper, scissors or camera.

An exemplary grasper is schematically illustrated in FIG. 1. A medical instrument 20 for use in this disclosure may have an elongate shaft suitable for passing through the introducer 10 and into a surgical site, or may be inserted into a carrying shaft (not shown) for better compatibility with the introducer 10.

The instrument drive assembly 22 is configured to incrementally advance the medical instrument 20 towards and through the distal end 16 of the introducer 10. The instrument drive assembly 22 may have an instrument drive actuator 24 and incremental drive linkages 26. A variety of instrument drive assemblies 22 may be suitable for use in the introducer 10. For example, the instrument drive assembly may be manually powered, electrically powered, pneumatically powered, hydraulically powered, or otherwise. Further, the instrument drive assembly be manually triggered, or may be automatic. Examples of different configurations for drive assembly 22 are depicted in FIGS. 2-4.

FIG. 2 depicts a first exemplary embodiment of the drive assembly 22, in which the drive assembly 22 is manually powered and manually triggered. A side, cross-sectional view of a part of the housing 12 is depicted. In this embodiment, actuator 24 includes manual trigger 44 pivotably coupled to handle 40 and arranged to abut a spring-biased tab 48. Examples of triggers could also be smaller (similar to that found on firearms), or could be a button near the grip site (similar to joysticks and computing gaming), or further could include a three-finger actuator, such as those used to actuate syringes. Other possible triggering mechanisms could be foot activated (such as with a pedal), or remotely activated (as in the case of robotic surgery where the activation would take place at a console).

As shown in FIG. 2, teeth on a linear rack 50 interlock with teeth on the tab 48. Both the tab 48 and the rack 50 are depicted with opposing angled teeth. The rack 50 is coupled to the shaft 21 of the medical instrument 20 with, for example, a releasable clip 54. A clamp or other type of coupling mechanism may be used in lieu of a clip 54. In an alternative arrangement, a proximal end or rack 50 could include an abutment that would contact the proximal end of medical instrument 20 to push the medical instrument 20 distally. Upon the trigger 44 being actuated towards the handle 40, as indicated by arrow 59, the trigger 44 pushes the tab 48 towards the distal end 16 of the introducer 10. The tab 48 compresses a spring 58 as it moves toward the distal end, and engages the angled teeth of the rack 50. The rack 50 and coupled medical instrument 20 are therefore pushed towards the distal end 16 of the introducer 10 with the tab 48. Upon release of the trigger 44, the trigger 44 and the spring-biased tab 48 return to their original positions due to pressure from the compressed spring 58. The angle of the teeth on the tab 48 and the rack 50, together with the pressure from the compressed spring 58, allow the tab 48 to disengage from the rack 50 when returning to its original position, leaving the rack 50 and the coupled medical instrument 20 in an incrementally advanced position. In this embodiment, an incremental advancement distance for the medical instrument 20 may be determined by, for example, the “throw” length and position of the trigger 44, the length and resistance to compression of the spring 58, and/or the size of the teeth on the tab 48 and the rack 50.

In addition or alternatively, drive assembly 22 may include a rotational drive component 23 (FIG. 2) for rotating the medical instrument 20. According to one aspect of the disclosure, the rotational drive component 23 may be actuated with trigger 44, however, it is understood the rotational drive component 23 may be separately actuatable. If associated with trigger 44, rotational drive component 23 may include any conventional structure for converting liner to rotational movement. Thus, according to one aspect of this disclosure, actuation of trigger 44 can actuate both incremental linear advancement and incremental rotation of medical instrument 20. Alternatively or additionally, the rotational drive component 23 may include, a manual knob, or other mechanism coupled to the medical instrument 20 to allow for selective rotation of the medical instrument 20 by the user. Rotating the medical instrument 20 during introduction assists in movement of the medical instrument 20 through the tissue. For example such rotation would assist the introduction of a trocar incorporating laparoscopic devices. The introduction of a trocar or other similar device provides a mini port that allows for the re-introduction of other medical instruments 20, such as flexible and semi-flexible catheters. When introducing a trocar with the introducer 10, a camera may be provided in the trocar lumen to provide for visualization during port placement.

FIG. 3A depicts a second embodiment of a manually powered and manually triggered instrument drive assembly 60. A side, cross-sectional view of an open frame housing 62 is depicted. A manual trigger 64 is coupled to a handle 66, and abuts an advancement bar 68. Handle 66 may have left and right parts 66 (only one shown) with the trigger 64 sandwiched therebetween. The advancement bar 68 includes an opening 70 for passage of the medical instrument 20, and is coupled to the housing 62 by an angling spring 74. Upon the trigger 64 being pulled proximally, as indicated by arrow 71, the advancement bar 68 has an end 76 that is pushed towards the distal end 78 of the housing 62 while a spring 74 restricts proximal movement of an end 79 of the advancement bar 68 opposite end 76. The angling of the advancement bar 68 causes the shaft 21 of the medical instrument 20 to be secured with the edges of the opening 70 in the advancement bar 68, thereby gripping the shaft 21 of the medical instrument 20 and advancing the instrument 20 toward the distal end 78. Upon release of the trigger 64, the advancement bar 68 loses securement on the medical instrument 20 and returns to its original position, leaving the medical instrument 20 in an incrementally advanced position. A brake bar 80, located at the proximal end 81 of the housing 62, also includes an opening 82 through which the shaft 21 of the medical instrument 20 passes. A spring 72 angles the brake bar 80 and opening 82 to provide a grip on the shaft 21 of the medical instrument 20 to prevent unintended movement of the medical instrument 20 away from the distal end 78, when the advancement bar 68 is not coupled to the shaft 21 of the medical instrument 20. The brake bar 80 may be pressed distally, compressing the brake spring 72 and eliminating the gripping action of the brake bar 80 on the shaft 21 of the medical instrument 20, allowing for a manual pushing or pulling movement of the medical instrument 20 when the advancement bar 68 is not gripping the shaft 21 of the medical instrument 20.

FIG. 3B depicts an introducer device 300 similar to the introducer device 10 of FIG. 1, and drive assembly 60 of FIG. 3A, with common reference numbers being used to identify the same or similar elements. Introducer device 300 includes a stroke limiter 310 having, for example, a threaded bolt 312 extending through brake bar 80 and brake spring 72, and threadingly engaging housing 62. Stroke limiter 310 has a distal end 314 (shown in dashed lines) that abuts a top end of trigger 64 to limit forward motion of the bottom end of trigger 64 (i.e., counter-clockwise motion of trigger 64 in FIG. 3B). Turning/rotating threaded bolt 312 controls the axial position of the distal end 314, which controls the stroke length of trigger 64, thus adjusting the movement of shaft 21 of medical instrument 20 for each trigger actuation. It is understood that stroke limiter 310 need not include the threaded bolt 312, but could include any other appropriate element or elements to limit the travel of trigger 64.

The introducer device 300 of FIG. 3B may also include a shaft securing device 318. Shaft securing device 318 may include a rotating plate that blocks the medical instrument 20 from being radially removed from housing 62. Housing 62 may include a series of aligned radial grooves 322 (located on the introducer device 300 opposite the side shown in FIG. 3B, and indicated with dashed lines) extending to a centerline of the device 300. Similarly aligned radial grooves are also included in brake bar 80 and advancement bar 68. The aligned radial grooves allow the medical instrument 20 to be radially inserted into and removed from housing 62. Shaft securing device 318 is rotatable about an axis parallel to medical instrument 20, to block or unblock a portion of radial groove 322 in housing 62. When unblocked, the medical instrument 20 can be introduced or removed from housing 62. When blocked, the shaft securing device 318 assists in ensuring that medical instrument 20 remains properly positioned within the radial grooves 322. It is understood that shaft securing device 318 can be any type of device that closes a portion of the radial grooves to help secure the medical device 20 in place.

The introducer device 300 of FIG. 3B may be configured to include a distal cap 330 similar to distal cap 18 of the introducer device 10 of FIG. 1, except that fluid conduit 34 may directly couple to distal cap 330 rather than run through housing 62. Thus, fluid conduit 34 may couple to the valve assembly 32 and fluid supply outside introducer device 300. It is understood that valve assembly 32 may alternatively be formed as part of distal cap, and can be controlled in any appropriate manner.

Distal cap 330 may be secured to housing 62 in any appropriate manner, such as the peg-in-groove coupling disclosed with respect to the introducer device of FIG. 1. Distal cap 330 may alternatively be coupled to housing 62 with a ball-and-socket type connection, such that the distal cap 330 may swivel and rotate with respect to housing 62. Such a ball-and-socket connection may include a ball element (not shown) protruding from housing 62, and a mating locking cap and end cap (not shown) on the proximal and distal sides of the ball, respectively (and/or vice versa). The locking and end caps can be, for example, threadingly engaged allow both sliding and locking of the ball between the locking and end caps. Alternatively, the locking cap can include a slot to limit movement of the distal cap 330 relative to the housing 62 only along the slot axis.

FIG. 3C depicts an introducer device 400 similar to the introducer device 300 of FIG. 3B, with common reference numbers being used to identify the same or similar elements. Introducer device 400 includes a drive rod 410 that is coupled to the medical instrument 20 via a releasable clip 412, similar to the coupling described with respect the embodiment of FIG. 2. Drive rod 410 is driven by an advancement bar 68 in a similar manner as the drive assembly 60 of FIG. 3A.

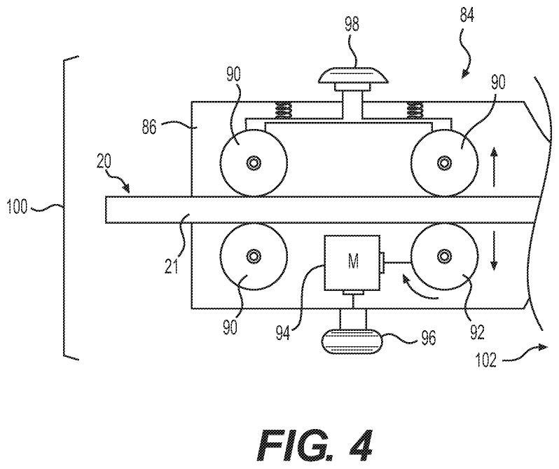

FIG. 4 depicts another alternative embodiment of the instrument drive assembly 22 of FIG. 1. This embodiment considers that the drive mechanism can be powered from a mains supply (at line voltage or stepped down by a transformer), battery power pack or other similar power supply to drive the instrument the requisite incremental distance. The drive mechanism also incorporates a safety override to prevent accidental over-introduction, examples of such sensing may occur from closed loop feedback, pressure sensing or other suitable methods. In this embodiment, an introducer 100 includes an automated drive assembly 84. The drive assembly 84 is schematically depicted in a cross-sectional side view in FIG. 4. The automated drive assembly 84 may be in a housing 86 that may split in half to facilitate loading or unloading of the medical instrument 20. A plurality of holding rollers 90 are configured to hold the medical instrument shaft 21 and allow for smooth advancement. At least one driving roller 92 is connected to a driving motor 94 which is controlled by an actuator 96. In FIG. 4, the actuator 96 is represented by a button, but may be in any form that can activate the driving motor 94. The rollers 90, 92 may be engaged or disengaged from the shaft of the medical instrument 20 by means of a roller engagement mechanism, controlled by, for example, a roller engagement button 98. Additional idler rollers (not shown) may be included to aid in keeping the shaft 21 of the medical instrument 20 appropriately positioned, and allow for smooth advancement of the medical instrument 20 towards a distal end 102 of the housing 86.

A variety of configurations of driving rollers, holding rollers and idler rollers are possible, depending on the medical instrument 20 intended for use in the housing 86. In this embodiment, the mechanism to measure or control the incremental advancement of the medical instrument 20 may be determined by, for example, a fixed time of delivery of power to the driving motor 94 each time the actuator button 96 is pressed. The fixed time may be set, for example, by an increment adjustor button or dial (not shown) on the device itself. Alternately, the actuator button 96 may directly activate the driving motor 94, such that the user may control the advancement distance by pressing the actuator button 96 for the desired amount of time. The driving motor 94 may be powered using a direct connection to an electrical outlet, or may be battery-operated to allow for more freedom of movement during use of the introducer 100. Further, the actuator button 96 may power both the driving motor and the fluid supply assembly 28, such that activating the actuator button 96 may release a desired amount of fluid and subsequently or substantially simultaneously advance the medical instrument 20. Even further, driving motor 94 may be reversible so that the medical instrument 20 may be withdrawn if desired.

The embodiment of FIG. 4 may also include a driving motor 94 with pressure-sensing capabilities. As such, the driving motor 94 may be able to sense the pressure acting on a medical instrument 20 as it progresses through tissue layers, for example, in the abdomen, to the peritoneal layer. This pressure sensing capability may have several uses. For example, a medical instrument 20 that is being incrementally inserted by a medical introducer 100 according to this disclosure will experience a rise in pressure while traversing the peritoneal layer. Once the medical instrument 20 passes through the peritoneal layer, the pressure on the medical instrument will reduce. By sensing these changes in pressure through the driving motor 94, an introducer 100 according to FIG. 4 may be able to sense that the medical instrument 20 is under little or no pressure and is therefore inserted into the abdominal cavity. As another example, pressure-sensing capabilities in the driving motor 94 could be used to sense when the medical instrument 20 has collided with tissue within the surgical site. An introducer 100 according to this embodiment may be linked to or equipped with an alert system, such as a display screen, a light, or an audible alert, to notify the user of the introducer 100 that the medical instrument 20 is inserted into the abdominal cavity, or that medical instrument 20 has collided with tissue within the surgical site. In another embodiment other sensors such as pH, oxygen monitoring, temperature monitoring and other such sensing could also be introduced using the introducer device.

Referring back to FIG. 1, and as noted above, introducer 10 according to the present disclosure may include a fluid supply assembly 28, comprising a pressurized fluid supply 30, an valve assembly 32, and a fluid conduit 34 from the valve assembly 32 to the distal end 16 of the introducer housing 12. The fluid supplied by the fluid supply assembly 28 may be any surgically compatible fluid, such as a non-combustible gas (e.g., carbon dioxide) or a sterile liquid (e.g., saline). In another embodiment the surgically compatible fluid could include therapeutic fluids/gels/other substances; some examples include local anesthetic agents, local pain relief and hemostasis substances.

Pressurized fluid supply 30 may be connected to the housing 12 through the valve assembly 32. The valve assembly 32 may comprise a manual control valve including a manual actuator 110, and a two-position valve body 33 biased toward a closed position (shown in FIG. 1). Upon actuation of the actuator 110 the valve body 33 moves to an open position and pressurized fluid from the fluid supply 30 enters the fluid conduit 34 and supplies pressurized fluid to the distal end 16 of the housing 12. The valve assembly 32 may be a manually-activated mechanical button as shown in FIG. 1, or may comprise an electronic valve assembly. The system may supply pressurized fluid under constant or variable pressure.

As best seen in FIGS. 1 and 5, fluid conduit 34 runs from the valve assembly 32 through the housing 12, to the distal end 16 of the housing 12, where it joins with a passage 38 in the distal cap 18. Alternatively, fluid conduit 34 could be formed with a separate tube (e.g., a rubber or plastic tubing) running outside the housing 12 and extending from valve assembly 32 to a connection at the distal end 16 of housing 12. In a further embodiment where the device is used outside the hospital (for example in battlefield hospital) there may also be a requirement for the device to include a self-contained gas (such as a CO₂ cartridge) or liquid supply. When air is used as a gas to assist with incising, a bulbous hand pump may be attached to the trigger 40. With each activation of the trigger 40 the gas is transmitted to the required location.

In one embodiment, towards the distal end 16 of introducer 10, the conduit 34 widens to become a conduit for both the instrument 20 and the pressurized fluid, such that the shaft 21 of the medical instrument 20 and the fluid pass through the same conduit 34 at the distal end 16. A seal 114, such as an o-ring seal, may be located at a proximal portion 35 of conduit 34, prevents fluid in the conduit from exiting the conduit 34 at the proximal entry point of the medical instrument 20 into the conduit 34. In addition or alternatively, the medical instrument may include a seal (not shown) about its shaft to assist in sealing the fluid passage. The fluid conduit 34 may be coaxial with the conduit for the medical instrument 20, as depicted in FIG. 5. Further, portions of fluid conduit 34 may be exterior to the housing 12 of the introducer 10, until it enters the housing 12 near the distal end 16 of the housing 12 and creates a conduit for both the medical instrument 20 and the pressurized fluid supply. This conduit may also surround the instrument and extend into the tissues.

As noted above, the introducer 10 according to the present disclosure also includes a distal cap 18 at the distal end 16 of the housing 12. The distal cap 18 may include a distal coupling end 36 for coupling with the outer tissue (e.g., skin) at a surgical site of a subject. The coupling end 36 may be configured to couple to the tissue of the surgical site in a manner that allows the user of the introducer 10 to pull the outer tissues up and away from the inner tissues. For example, the coupling end 36 may be coated with a waterproof biocompatible adhesive suitable for attaching to skin. As another example, the coupling end 36 may be coupled to tissue with one or more manually-operated tissue clamps positioned around the flange 37. As a third example, the coupling end 36 may include one or more openings 111 fluidly connected to a suction supply 112 to create a suction-type connection with the skin. Openings 111 in coupling end 36 may include suction cups to better isolate the suction pressure. While coupling end 36 is shown in FIG. 1 as being circular, it is understood that other shapes are possible, such as an “X” shape with each branch of the “X” having a suction opening 111. Alternatively, the coupling end may include a series of ring shaped grooves separated by protruding rings forming a bulls-eye configuration. Alternating rings could be connected with suction supply 112. By applying suction to separate sections of the coupling end 36, the potential for the leakage of positive or negative pressure is lessened. According to one aspect, applying additional vacuum bursts with each actuation of trigger 40 would allow better contact with the skin and less potential for accidental detachment.

The distal cap 18 of the introducer 10 also may include a passage 38 for receiving the medical instrument 20 as it passes out an exit port at the distal end 16 of the introducer 10, as well as the fluid supply from the fluid supply conduit 34. For example, FIG. 5 depicts a distal cap 18 with a centered passage 38 that continues from the fluid conduit 34 in the housing 12, and that ends in a distal opening of the introducer 10 through which both the medical instrument 20 and fluid from the fluid supply 30 may pass to exit the introducer 10. A further embodiment of this system is that it could also be developed as an attachment for robotic devices where for example the robotic arm approaches the patient. A suction end 36 that attaches to the skin and instruments are automatically introduced.

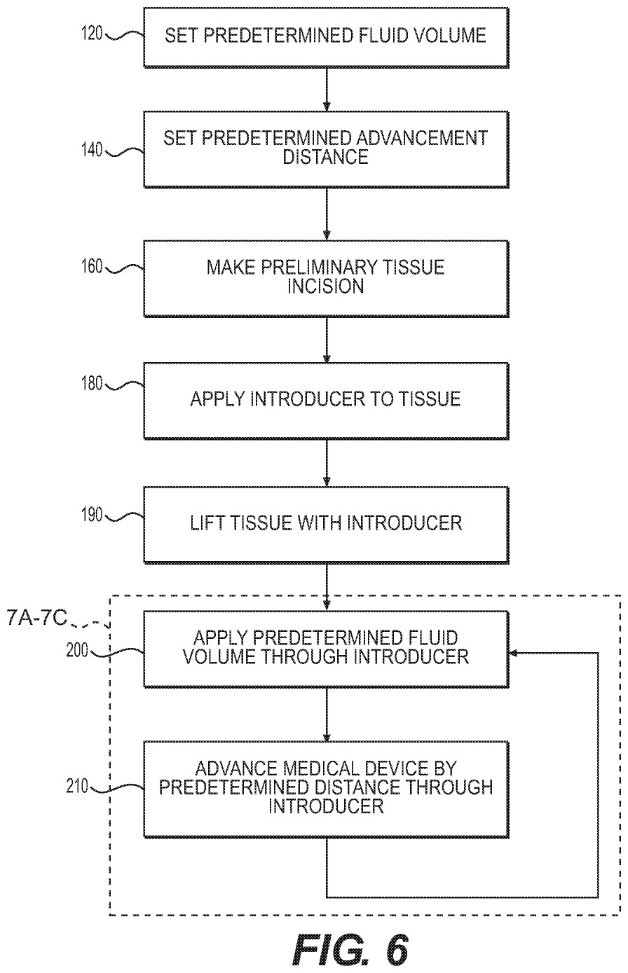

FIG. 6 depicts a method for advancing a medical instrument into a surgical site in a controlled manner. According to a first step 120, a predetermined volume of fluid may be set, and may be based on parameters relevant to the surgery, surgeon and/or patient. The volume of fluid may be preset, or may be variably set by the operator. As noted above, the fluid may be a biocompatible noncombustible gas known in the art (e.g., carbon dioxide), or may be a biocompatible liquid. In step 140, a predetermined advancement distance for the medical instrument 20 into the surgical site may also be set, and may be based on parameters relevant to the surgery, surgeon and/or patient, and/or on the predetermined volume of fluid. The advancement distance may also be preset, or may be variably set by the operator. For example, in some laparoscopic procedures, a predetermined advancement distance may be between 2 and 4 millimeters. The distance advanced may also be regulated by the pressure (or other) sensor at the tip of the instrument. The addition of a monitoring imaging or sensory device (such as an ultrasound or a chip) able to identify intra-abdominal viscera (such as intestine, spleen, liver) and blood vessels, and to distinguish it from the abdominal wall, would provide for a more safe device since it would incorporate an automatic safety mechanism to avoid further advances of the instrument if such structures are not displaced by the bursts of fluid/air.

In step 160, a preliminary incision may be made in the tissue at the instrument introduction site. The preliminary incision may be made by using a small blade on the medical instrument introducer 10, or optionally using a separate, suitable surgical instrument known in the art. Alternatively small blade or lancet may be incorporated in the introducer that activated to make the initial incision (similar to mechanisms used to obtain a drop of blood to measure glucose levels in diabetic patients). Another variation would include having the blade advance together with the instrument, and cutting the tissues with each triggering.

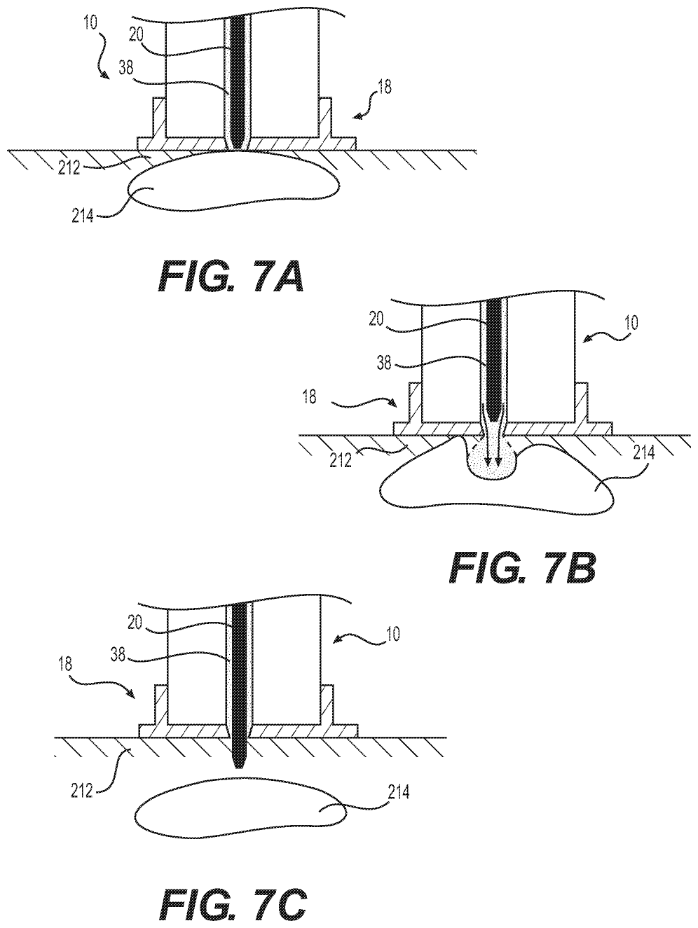

The introducer 10 may then be applied to the tissue at the instrument introduction site (step 180). For example, FIG. 7A depicts the introducer 10 being applied to tissue at a surgical site. Application of the introducer 10 to the tissue at the site may include coupling the introducer to the tissue at the instrument introduction site by means of an adhesive such as a biocompatible adhesive, medical tissue clips, or other means known in the art, as described above. Once secured to the tissue, the introducer 10 may be used to lift the outer tissue at the introduction site away from the inner tissue (step 190), allowing for more space to be created underneath the outer tissue.

A predetermined volume of fluid may then be supplied through the introducer 10 into the preliminary incision at the instrument introduction site (step 200). For example, FIG. 7B depicts the introduction of fluid through the medical introducer 10 into the surgical site. The medical instrument 20 may then be advanced by the previously set predetermined distance through the introducer 10 and the preliminary incision (step 210). Step 210 may be carried out after completion of supplying of the predetermined volume of fluid (step 200) or may be carried out merely after initiation of the supply of fluid. For example, FIG. 7C depicts the advancement of a medical instrument 20 through the medical instrument introducer 10 and the preliminary incision. Following insertion of the medical instrument 20 into the surgical site by the predetermined distance, the predetermined volume of fluid may be reapplied through the introducer and the medical instrument may be further advanced by the predetermined distance repeatedly, until the medical instrument is inserted to the desired depth and/or position. It is understood that the present disclosure is not limited to a method requiring all of the steps of FIG. 6, but rather, certain identified steps may be omitted, and/or other steps added.

FIGS. 7A-7C further depict a method of introducing a medical instrument 20 into a surgical site using an introducer 10. The distal cap 18 of the introducer 10 is brought to a preliminary incision in a patient's outer tissue 212 at a surgical site. The coupling end of the distal cap 18 is brought into contact with the outer tissue 212. As previously described, the coupling end may be adhered or affixed to the outer tissue 212 by a variety of means. Referring to FIG. 7B, a pre-set amount of fluid is introduced through the central passage 38 into the preliminary incision. The introduced fluid may create a new space between the outer tissue 212 and underlying viscera 214 adhering to the outer tissue 212, as depicted in FIG. 7B, or may enlarge a preexisting space between the outer tissue 212 and underlying viscera 214. Referring to FIG. 7C, the medical instrument 20 is then advanced by a pre-set distance into the space created or maintained by the fluid. Variations on the above-described introducer and method will be evident to those of ordinary skill in the art.

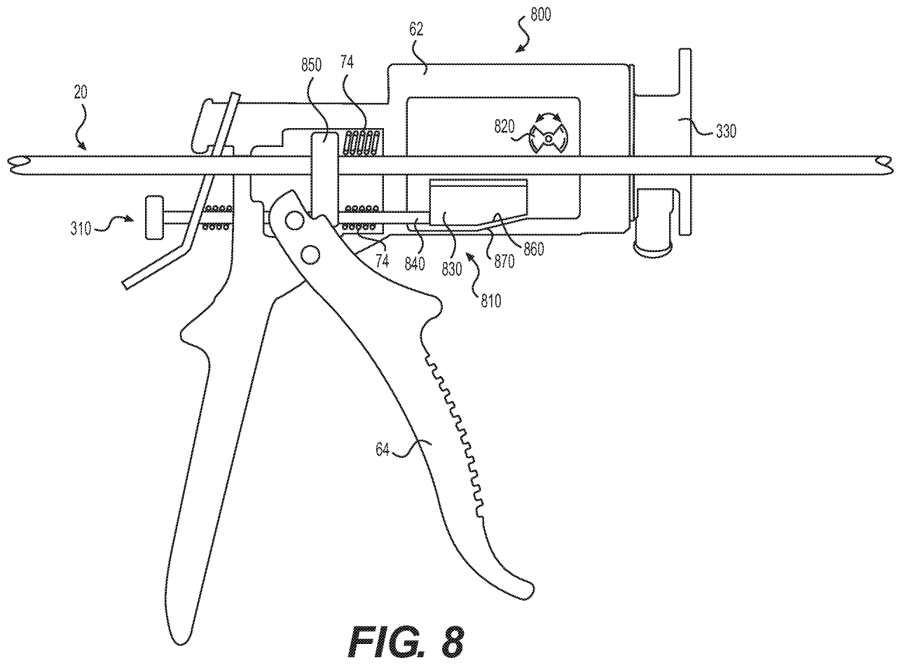

FIG. 8 depicts an introducer device 800 similar to the introducer device 300 of FIG. 3B, with common reference numbers being used to identify the same or similar elements. Introducer device 800 includes a drive assembly 810 having a rotating cam 820 and a clamp 830 coupled for movement with trigger 64. The rotating claim 820 may be rotated into contact with the medical instrument 20 by manual manipulation via a dial or button located outside the housing 62.

Clamp 830 is coupled to trigger 64 through a rod 840 and plate 850 rigidly coupled to clamp 830. Upon actuation of trigger 64, a top end of trigger 64 contacts and moves plate 850 distally, which in turn moves rod 840 and clamp 830 distally. Upon movement of the clamp 830 distally, a ramp 860 of the clamp 830 slides on a ramp 870 of housing 62, and urges the clamp 830 toward medical instrument 20. The clamp 830 engages and moves medical instrument 20 if the rotating cam 820 has been moved into contact with the medical instrument 20. However, if the rotating cam 820 is in the position shown in FIG. 8, then distal (or proximal) movement of clamp 830 will not move medical instrument 20. Thus, the rotational position of the rotating cam 820 dictates whether the clamp 830 moves the medical instrument 20. In such an arrangement, the rotating cam 820 can be positioned to move medical instrument 20 distally or proximally depending on whether the trigger is moving plate 850 distally, or allowing plate 850 to move proximally due to springs 74.

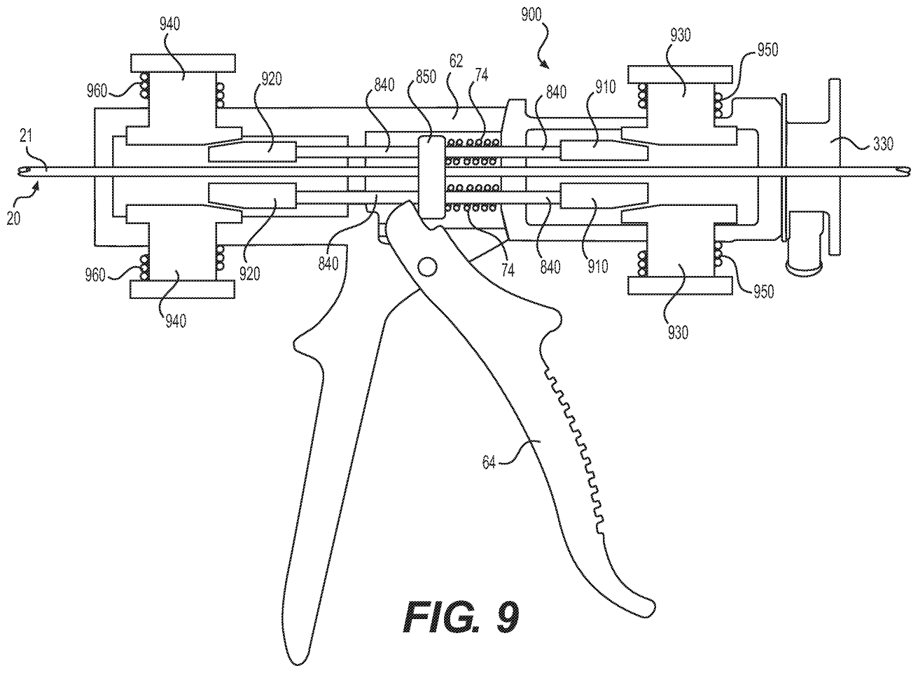

FIG. 9 depicts an introducer device 900 similar to the introducer device 800 of FIG. 8, with common reference numbers being used to identify the same or similar elements. Introducer device 900 includes a plurality of forward motion cams 930, and a plurality of rearward motion cams 940. The forward and rearward motion cams 930 and 940 are secured to housing 62, and movable radially with respect to the housing 62. Springs 950 and 960 urge forward and rearward motion cams 930 and 940 radially outward. A plurality of clamps 910 and 920 are rigidly coupled to rods 840 and plate 850 to move with the actuation and release of trigger 64. Upon proximal or distal movement of plate 850, rods 840, and clamps 910 and 920, forward or rearward motion cams may be actuated manually (depressed radially) to secure clamps 910 or 920 on medical instrument 20, and thereby move medical instrument 20. Thus, depending on when the cams 930 or 940 are actuated during the movement of plate 850, the medical instrument 20 can be moved proximally and/or distally.

The introducer device 900 may also include a stroke limiter (not shown) protruding inwardly from the housing 62 to limit movement of plate 850. Further, releasable locking mechanisms (not shown) may be incorporated into forward and rearward motion cams 930 and 940 to further control movement of the medical instrument 20.

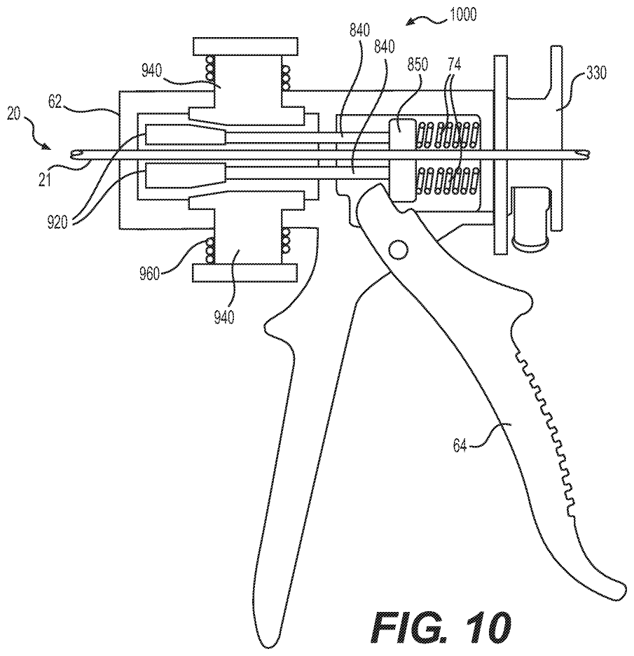

FIG. 10 depicts an introducer device 1000 similar to the introducer device 900 of FIG. 9, with common reference numbers being used to identify the same or similar elements. Introducer device 900 includes a plurality of motion cams 940 secured to housing 62, and movable radially with respect to the housing 62. Springs 960 urge motion cams 940 radially outward. A plurality of clamps 920 are rigidly coupled to rods 840 and plate 850 to move with the actuation and release of trigger 64. Upon proximal or distal movement of plate 850, rods 840, and clamps 920, motion cams 940 may be actuated manually (depressed radially) to secure clamps 920 on medical instrument 20, and thereby move medical instrument 20. Thus, depending on when the cams 940 are actuated during the movement of plate 850, the medical instrument 20 can be moved proximally and/or distally.

Further additions to and variations on the medical introducers and method disclosed herein are possible. For example, the introducers in any of the embodiments described herein may include an imaging device, such as an ultrasound probe, to allow for even safer incremental advancement of a medical instrument 20. Additionally, any of the above embodiments may further include a safety catch, valve, or switch, to prevent accidental injection of fluid, overpressurization, or advancement of the medical instrument beyond what is desired. Further, any of the above embodiments may be used or performed in combination with added lubrication for easier access to the surgical site, provided by flushing saline through the introducer and into the site, or by the separate addition of sterile lubrication to the site. The above may also provide thermal changes, such as freezing and cauterizing. Introducers according to this disclosure may be single-use devices, or may be created so as to be reusable. The embodiments described herein can be used independently of other laparoscopic introduction tools, such as trocars or ports, or can optionally be used in combination with trocars, ports and/or other surgical instruments.

While this patent introduces a new version of technology, the introducer 10 will also complement existing laparoscopic techniques. For example, in cases where additional instruments may be required or would make the procedure simpler or quicker, introducing such instruments with the introducer 10 would make it more acceptable to the surgeon (and of more rapid implementation) since no additional ports will be required.

The invention according to this disclosure may exhibit a variety of features. For example, a controlled burst of fluid, when introduced in the abdominal cavity, may dissect away potential viscera or adhesions from the abdominal wall as well as from the instrument 20. This dissection may occur with or without a pneumoperitoneum in place; in the latter case, obviating the need for a pneumoperitoneum and increasing the safety of the surgery to the patient. The controlled burst of fluid may also decrease resistance imposed by the abdominal wall when attempting to reach difficult-to-access sites. Furthermore, the introduction of a pre-set volume of fluid and the advancement of a medical instrument 20 by a predetermined distance using the introducers and methods described herein may avoid uncontrolled or accidental pushing of a medical instrument into the abdominal cavity (or other surgical site), thus avoiding associated injuries to viscera and structures in the area of the surgery.

The devices and methods described herein also may result in the elimination of traditional ports and trocars for laparoscopic procedures, allowing for, for example, flexibility in surgical approach. Where a surgeon might traditionally have been limited to a small set number of immobile ports for a laparoscopic procedure, the devices and methods described herein allow for the creation of numerous ports in various positions, allowing for easier surgical access to desired sites with minimal added strain and risk on the patient. The elimination of port restrictions may also decrease interference, pushing, or collisions between various instruments being used simultaneously in the surgical field. The elimination of port restrictions may make laparoscopic techniques easier and safer, the approach could also facilitate both therapeutic and diagnostic procedures. The present system and method using fluid dissection may reduce the number of port scars associated with larger, set ports. Also, the present system and method may reduce the occurrence of hernias developing at the instrument (or trocar) insertion points, and decrease trauma to the abdominal wall. Additionally, the elimination of a need for a large port or trocar enables the use of smaller diameter medical tools (i.e., only the medical instrument and no surrounding port or trocar) having to be inserted into the surgical site. The insertion of a smaller instrument, and lower numbers of instruments, may mean a lower risk of injury to the patient, a less invasive surgical procedure, and/or a lower cost procedure. The insertion of a smaller instrument may also mean that once the medical instrument is withdrawn, no closure of a large port-created wound is required.

Another feature of the devices and methods herein is that they may expand the potential use scenarios for laparoscopic medical instruments and procedures. For example, a decrease in the complexity of laparoscopic surgeries caused by the elimination of a need for set ports may allow for more advanced, and greater use of, diagnostic laparoscopy. It is also envisaged that future pre-identified laparoscopic procedures could be completed with local anesthesia at locations previously not amenable to these techniques, with a decreased risk of infection, with portable single use, (sterile, disposable devices) with the potential to use imaging devices such as for example ultrasound and cameras used in conjunction with cell phones, laptops or portable computer tablet type devices. Laparoscopy using the devices and methods disclosed herein could be expanded to the diagnosis of various pathologies in intensive (critical) care units, emergency rooms by surgeons and emergency room physicians, in battlefield settings, in rural areas, or in telemedicine, for example. Diagnostic laparoscopy using these devices could be performed as a bedside procedure, under local anesthesia, (especially if spraying the anesthetic agent at the site of the intervention and over the peritoneal surfaces), as only a small sterile field may be required, and closure of the surgical wound may be accomplished simply by withdrawing the medical instrument or instruments, without the need for sutures. The devices and methods disclosed herein could also be used to add laparoscopy to interventional radiology procedures, allowing for hybrid imaging/laparoscopic approaches to radiology, and an expanded spectrum of diagnostic and/or therapeutic interventions. Similarly, laparoscopy could be added to hybrid procedures, endoscopic procedures such as colonoscopies, gastro-duodenoscopies, and ERCP, allowing for a combined endo-exo visceral approach to better outline pathologies and obtain tissue samples. The device could also be considered for use in various interventional radiology procedures, fertility treatment procedures, hybrid procedures (such as endoscopy, interventional radiology), nerve block and other various pain management procedures or could also be considered for cardio-thoracic or orthopedic procedures. The instruments may also be compatible to be used in conjunction with Magnetic resonance, CAT (CT) Scan, Positron Emission Tomography (P.E.T. Scan), Ultrasound and other imaging systems.

The devices and methods disclosed herein can also be used as a complement to traditional laparoscopic instruments, as portless instruments introduced according to this disclosure could facilitate the action of classic laparoscopic instruments and techniques.

A further feature of the devices and methods described herein is that they may be adaptable to a wide variety of cases and procedures beyond laparoscopic procedures, such as pediatric interventions, ob/gyn procedures, and neurologic procedures (such as operations in small fields, procurement of cells, and spinal access procedures). The devices and methods herein may also be applicable in veterinary procedures. Introducers and methods according to this disclosure may be adapted to introduce a medical instrument into the abdominal cavity, or elsewhere in the body, such as into the reproductive system or the circulatory system. Further, as explained above, the disclosed introducers are configured to be used with a variety of different instruments, and thus is not limited to only introducing a particular instrument.

Other embodiments of the present disclosure will be apparent to those skilled in the art from consideration of the specification and practice of the embodiments disclosed herein. While various examples provided herein illustrate specific types of introducers and methods, one of ordinary skill in the art will recognize that other configurations of a medical instrument introducer, and variations upon the methods described, also may be used. For example, the introducer 10 could insert the medical instrument 20 without the positive pressure or suction pressure described above. Further, any features of an embodiment disclosed herein may be incorporated into any other embodiment.

What is claimed is:

1. A method of inserting an instrument into a patient, comprising:
supplying pressurized fluid to an introducer to an instrument introduction site; and
advancing an instrument located in the introducer through the introduction site after an initiation of supplying of pressurized fluid.

2. The method of claim 1, wherein the supplying pressurized fluid includes introducing a preset volume of fluid to the introduction site, and advancing the instrument includes advancing the instrument a preset distance.

3. The method of claim 1, wherein the supplying pressurized fluid and advancing the instrument are provided through a same distal opening in the introducer.

4. The method of claim 1, wherein the method further includes adhesively attaching the introducer to the instrument introduction site.

5. The method of claim 4, wherein the method further includes lifting tissue at the instrument introduction site prior to supplying the pressurized fluid.

6. The method of claim 1, further including additionally advancing the instrument by repeatedly supplying pressurized fluid through the introducer to the instrument introduction site and stepwise advancing the instrument through the introduction site after each supplying of pressurized fluid, until the instrument has attained a desired position.

7. The method of claim 1, further including making a tissue incision at the instrument introduction site prior to the supplying of pressurized fluid.

8. The method of claim 1, wherein the pressurized fluid is one of carbon dioxide or a saline solution.

9. The method of claim 1, wherein the instrument introduction site is a location in the abdominal wall, and the instrument is a laparoscopic instrument.

10. A medical instrument introducer, comprising:
a housing having a proximal end and a distal end, and an instrument support supporting an instrument from the proximal end to the distal end; and
a instrument drive assembly having a drive actuator to incrementally advance the instrument through the housing.

11. The medical instrument introducer of claim 10, further including a pressurized fluid supply assembly having a pressurized fluid supply and a valve assembly for controlling the supply of pressurized fluid supplied through the introducer.

12. The medical instrument introducer of claim 11, wherein the pressurized fluid supply assembly further includes a fluid conduit coupled to a distal end of the introducer.

13. The medical instrument introducer of claim 12, wherein the distal end of the introducer includes an instrument conduit in fluid communication with the fluid conduit.

14. The medical instrument introducer of claim 12, wherein the pressurized fluid supply assembly includes a fluid supply actuator, and the fluid supply actuator and drive actuator are separate manual actuators.

15. The medical instrument introducer of claim 10, wherein the instrument drive assembly includes incremental drive linkages coupled to the drive actuator.

16. The medical instrument introducer of claim 15, further including a stroke limiter for adjusting the incremental advancement.

17. The medical instrument introducer of claim 10, further including a plurality of radial grooves configured to allow radial insertion or removal of the medical instrument.

18. The medical instrument introducer of claim 10, further including a removable distal cap.

19. A medical instrument introducer, comprising:
a housing having a proximal end and a distal end, and an instrument support supporting an instrument from the proximal end to the distal end;
a pressurized fluid supply assembly;
a instrument drive assembly having a drive actuator to incrementally advance the instrument through the housing; and
an exit port at the distal end of the housing, the exit port receiving both the instrument and pressurized fluid from the pressurized fluid supply assembly.

20. The medical instrument introducer of claim 19, wherein the pressurized fluid supply assembly includes a pressurized fluid supply and a valve assembly for controlling the supply of pressurized fluid supplied through the introducer.
